(19) United States
(12) Patent Application Publication
(10) Pub. No.: US 2026/0364626 A1
(43) Pub. Date: Nov. 19, 2026

(54) ULTRASONIC ATOMIZER USING A LIQUID BOTTLE

(71) Applicant: DONGGUAN YIH TEH ELECTRIC PRODUCTS CO., LTD., DongGuan City (CN)

(72) Inventor: Ming Jen Hsiao, Miaoli County (TW)

(21) Appl. No.: 19/636,861

(22) Filed: Apr. 1, 2026

Related U.S. Application Data
(63) Continuation-in-part of application No. 18/937,160, filed on Nov. 5, 2024.

(51) Int. Cl. B05B 17/00; A61M 11/00; A61M 21/00; B05B 17/06; B65D 1/02; B65D 83/00

(52) U.S. Cl. CPC B05B 17/0646; A61M 11/005; A61M 21/00; B05B 17/0684; B65D 1/0246; B65D 83/00; A61M 2021/0016

(57) ABSTRACT

An ultrasonic atomizer using a liquid bottle. The ultrasonic atomizer comprises a power plug, a control circuit, and an atomizing module. the atomizing module is configured to be combined with a liquid bottle, the power plug is rotatably assembled to the atomizing module so that its angle can be changed, and is configured to be coupled to an electrical socket in various different orientations, such that the atomizer and the liquid bottle used therewith can be maintained vertically upward for atomization during use, a piezoelectric vibration element of the atomizer module is configured to be in continuous and effective contact with the liquid at a free end of a liquid-conducting element of the liquid bottle (for example, essential oil or aromatic liquid) to generate fragrance.

This application is a Continuation-in-Part of application Ser. No. 18/937,160 filed Nov. 5, 2024.

### BACKGROUND OF THE INVENTION

### 1. Field of the Invention

The present invention relates to an atomizing device, and more particularly to an ultrasonic atomizer using a liquid bottle.

### 2. Description of the Related Art

A conventional aroma diffuser uses an aromatic liquid bottle filled with aromatic liquid to emit fragrance molecules. A liquid-conducting element (wick) for absorbing liquid used in the aromatic liquid bottle is assembled for use in atomization by a piezoelectric vibration element of the aroma diffuser. The liquid-conducting element (wick) tends to have a loosening problem and is not easy to contact the piezoelectric vibration element, and therefore the atomization effect is poor or not continuous.

Furthermore, because liquid-conducting elements combined with different liquid bottles may differ in length, material, or deformation, the liquid at the free end thereof (such as essential oil or aromatic liquid) cannot be reliably transmitted to the piezoelectric vibration element for atomization. The liquid-conducting element, for example, is a soft liquid-absorbing rod made of cotton wick, paper rod, foam, or a porous liquid-conducting element, and is prone to deformation, especially after absorbing liquid, the side of the free end of the liquid-conducting element may tilt or loosen due to deformation, such that a connection gap between the free end of the liquid-conducting element and the piezoelectric vibration element becomes larger or the contact therebetween becomes incomplete, thereby resulting in insufficient transmission of liquid from the free end of the liquid-conducting element to the piezoelectric vibration element, making it difficult to maintain the liquid film thickness at the atomization interface, and causing some problems such as failure or abnormality of the atomizer function.

On the other hand, an essential oil bottle contains only a very small amount of essential oil and the price thereof is high. In order to allow precious essential oil to be transmitted through an essential-oil carrier to the piezoelectric vibration element for atomization, it is necessary to design an excellent essential-oil atomization passage to guide the essential oil absorbed by the essential-oil carrier to the piezoelectric vibration element for atomization without loss, and to allow the fine essential-oil droplets after atomization to diffuse and contact air into the ambient air to generate fragrance without wasting the essential oil.

### SUMMARY OF THE INVENTION

In view of the above problems of the conventional art, the present invention provides an ultrasonic atomizer using a liquid bottle, which can continuously and effectively atomize liquid.

According to an embodiment of the ultrasonic atomizer using a liquid bottle of the present invention, the ultrasonic atomizer using a liquid bottle, comprising a power plug, a control circuit, and an atomizing module, wherein the atomizing module comprises a piezoelectric vibration module, a mist-guiding tube, a hollow housing, a connecting tube, and an assembling seat, wherein the mist-guiding tube comprises an mist outlet on the upper side thereof and an inwardly protruding tube formed by downward protrusion from the inner side of the mist outlet, and the hollow housing is formed with a first opening at the upper side thereof and a second opening at the lower side thereof, wherein one side of the mist-guiding tube at the mist outlet is movably coupled in the first opening in the hollow housing, wherein the piezoelectric vibration module comprises a piezoelectric vibration element having plurality of micro-holes formed thereon, the piezoelectric vibration module is coupled in the mist-guiding tube, the upper side of the piezoelectric vibration module is connected to the free end of the inwardly protruding tube, the upper side of the connecting tube is coupled in the lower side of the mist-guiding tube, and the upper free end of the connecting tube is connected to the lower side of the piezoelectric vibration module, whereby the plurality of micro-holes of the piezoelectric vibration element, the inwardly protruding tube, the mist outlet, and the connecting tube form a communicating passage, wherein the assembling seat is coupled in the second opening, and the lower side of the connecting tube is movably connected to the upper side of the assembling seat, wherein the power plug is coupled to one side of the hollow housing, the power plug is electrically connected to the control circuit, the control circuit is electrically connected to the piezoelectric vibration element of the piezoelectric vibration module, wherein the control circuit comprises an oscillator circuit and a driver circuit.

According to an embodiment of the ultrasonic atomizer using a liquid bottle of the present invention, the ultrasonic atomizer using a liquid bottle is used with a liquid bottle, the liquid bottle being detachably coupled to the assembling seat, the liquid bottle being, for example, an aromatic liquid bottle or an essential oil bottle, the liquid bottle having a bottle opening and a liquid-conducting element, one side of the liquid-conducting element being disposed in the liquid within the liquid bottle, and another side thereof extending outward from the bottle opening and passing through the connecting tube, the free end of the liquid-conducting element protruding from the upper side of the connecting tube so as to be connected the lower side of the piezoelectric vibration element, the liquid-conducting element having, for example, a porous structure or capillary action and being configured to transport liquid from within the liquid bottle to the free end of the liquid-conducting element exposed outside the bottle opening, so as to provide liquid (such as essential oil or aromatic liquid) for atomization by the piezoelectric vibration element, by virtue of one side of the mist-guiding tube at the mist outlet being movably coupled in the first opening in the hollow housing, the upper side of the connecting tube being coupled in the lower side of the mist-guiding tube, and the lower side of the connecting tube being movably connected to the upper side of the assembling seat, the mist-guiding tube and the connecting tube are subjected to a downward gravitational force under the driving action of gravity, and at the same time the free end of the inwardly protruding tube is driven to push downward against the piezoelectric vibration element of the piezoelectric vibration module to move downward and abut against the free end of the liquid-conducting element, so as to solve the problem that, due to differences in length, different materials, or possible deformation and loosening of the liquid-conducting elements of different liquid bottles, the liquid at the free end thereof (such as essential oil or aromatic liquid) cannot be reliably transmitted to the piezoelectric vibration element for atomization. The ultrasonic atomizer using a liquid bottle of the present invention can continuously and effectively transmit the liquid at the free end of the liquid bottle to the piezoelectric vibration element for atomization.

### BRIEF DESCRIPTION OF THE DRAWINGS

FIG. 1 is a perspective view of an embodiment of the ultrasonic atomizer using a liquid bottle according to the present invention.

FIG. 2 is an exploded view of FIG. 1.

FIG. 3 is another exploded view of FIG. 1.

FIG. 4 is a front sectional view of the embodiment of the ultrasonic atomizer using a liquid bottle according to FIG. 1 of the present invention.

FIG. 5 is a sectional view of the power plug AA according to FIG. 4.

FIG. 6 is a schematic view according to FIG. 5, showing that, when the power plug is rotated by 90 degrees, the protrusion of the protruding rail is movably engaged with the stoppers of the concave rail.

FIG. 7 is a schematic view according to FIG. 5, showing that, when the power plug is rotated by 180 degrees, the protrusion of the protruding rail is movably engaged with the stoppers of the concave rail.

FIG. 8 is a schematic view according to FIG. 5, showing that, when the power plug is rotated by 270 degrees, the protrusion of the protruding rail is movably engaged with the stoppers of the concave rail.

### DETAILED DESCRIPTION OF THE PREFERRED EMBODIMENT

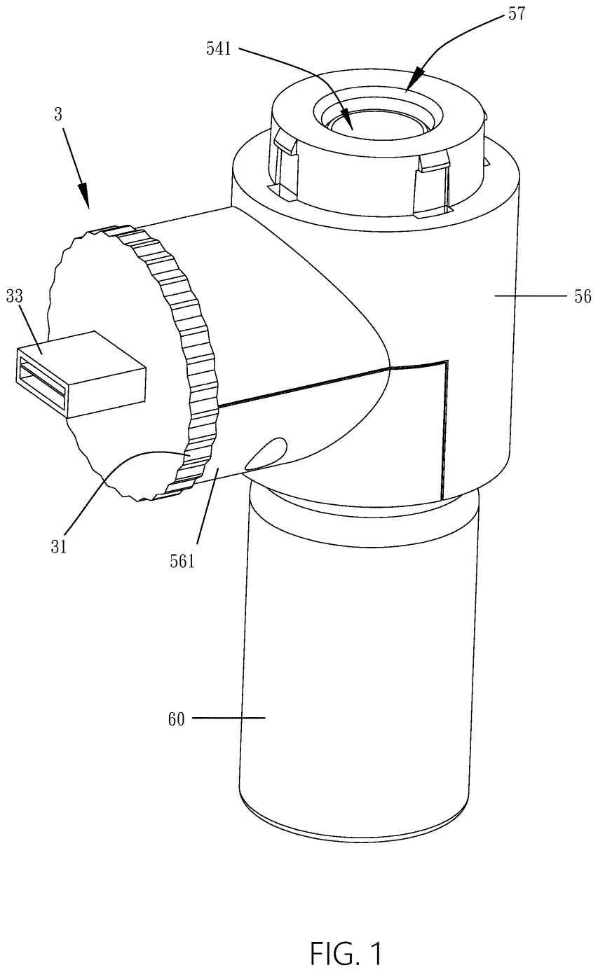

Please refer to FIGS. 1 to 4, In some embodiments of the ultrasonic atomizer using a liquid bottle of the present invention, an ultrasonic atomizer using a liquid bottle, comprising a power plug 3, a control circuit 30, and an atomizing module 5, wherein the atomizing module 5 comprises a piezoelectric vibration module 51, a mist-guiding tube 54, a hollow housing 56, a connecting tube 58, and an assembling seat 50, wherein the mist-guiding tube 54 comprises an mist outlet 541 on the upper side thereof and an inwardly protruding tube 543 formed by downward protrusion from the inner side of the mist outlet 541, and the hollow housing 56 is formed with a first opening 57 at the upper side thereof and a second opening 59 at the lower side thereof, wherein one side of the mist-guiding tube 54 at the mist outlet 541 is movably coupled in the first opening 57 in the hollow housing, wherein the piezoelectric vibration module comprises a piezoelectric vibration element 511 having plurality of micro-holes 513 formed thereon, the piezoelectric vibration module 51 is coupled in the mist-guiding tube 54, the upper side of the piezoelectric vibration module 51 is connected to the free end of the inwardly protruding tube 543, the upper side of the connecting tube 58 is coupled in the lower side of the mist-guiding tube 54, and the upper free end of the connecting tube 58 is connected to the lower side of the piezoelectric vibration module 51, whereby the plurality of micro-holes 513 of the piezoelectric vibration element 511, the inwardly protruding tube 543, the mist outlet 541, and the connecting tube 58 form a communicating passage, wherein the assembling seat 50 is coupled in the second opening 59, and the lower side of the connecting tube 58 is movably connected to the upper side of the assembling seat 50, wherein the power plug 3 is coupled to one side of the hollow housing 56, the power plug 3 is electrically connected to the control circuit 30, the control circuit 30 is electrically connected to the piezoelectric vibration element 511 of the piezoelectric vibration module 51. Wherein the control circuit 30 comprises an oscillator circuit and a driver circuit.

According to an embodiment of the ultrasonic atomizer using a liquid bottle of the present invention, the ultrasonic atomizer using a liquid bottle comprises a liquid bottle 60 for use therewith, the liquid bottle 60 being detachably coupled to the assembling seat 50, for example by screwing, the liquid bottle 60 being, for example, an aromatic liquid bottle or an essential oil bottle, the liquid bottle 60 having a bottle opening 67 and a liquid-conducting element 63, one side of the liquid-conducting element 63 being disposed in the liquid within the liquid bottle 60, and another side thereof extending outward from the bottle opening 67 and passing through the connecting tube 58, the free end 631 of the liquid-conducting element 63 protruding from an upper side of the connecting tube 58 so as to be connected the lower side of the piezoelectric vibration element 511, the liquid-conducting element 63 having, for example, a porous structure or capillary action and being configured to transport liquid from within the liquid bottle 60 to the free end 631 of the liquid-conducting element 63 exposed outside the bottle opening 67, so as to provide liquid (such as essential oil or aromatic liquid) for atomization by the piezoelectric vibration element 511, by virtue of one side of the mist-guiding tube 54 at the mist outlet 541 being movably coupled in the first opening 57 in the hollow housing 56, the upper side of the connecting tube 58 being coupled in the lower side of the mist-guiding tube 54, and the lower side of the connecting tube 58 being movably connected to the upper side of the assembling seat 50, the mist-guiding tube 54 and the connecting tube 58 are subjected to a downward gravitational force under the driving action of gravity, and at the same time the free end of the inwardly protruding tube 543 is driven to push downward against the piezoelectric vibration element 511 of the piezoelectric vibration module 51 to move downward and abut against the free end 631 of the liquid-conducting element 63, so as to solve the problem that, due to differences in length, different materials, or possible deformation and loosening of the liquid-conducting element 63 of different liquid bottles, the liquid at the free end 631 of the liquid-conducting element 63 thereof (such as essential oil or aromatic liquid) cannot be reliably transmitted to the piezoelectric vibration element 511 for atomization.

In some embodiments, the above-mentioned liquid-conducting element 63 extends from the bottle opening 67 of the liquid bottles 60 and passes through the connecting tube 58, and the free end 631 of the liquid-conducting element 63 protrudes from the connecting tube 58 and is connected the lower side of the piezoelectric vibration element 511. The liquid-conducting element 63 may be any one of a wick, a PET fiber rod, a paper rod, a foam rod, or a porous liquid-conducting element, and may be made of a material having a porous structure or capillary action. A soft liquid-absorbing rod made of such material is prone to tilting or deformation, especially after absorbing liquid, the side of the free end 631 of the liquid-conducting element 63 may deform and loosen, such that a connection gap between the free end 631 of the liquid-conducting element 63 and the piezoelectric vibration element 511 becomes larger or the contact therebetween becomes incomplete, thereby resulting in insufficient transmission of liquid from the free end 631 of the liquid-conducting element 63 to the piezoelectric vibration element 511 and making it difficult to maintain a liquid film thickness at the atomization interface between the free end of the liquid-conducting element 63 and the piezoelectric vibration element 511. The liquid-conducting element 63 may pass through the connecting tube 58 after protruding from the bottle opening, and the connecting tube 58 is configured to stably support the liquid-conducting element 63 and the free end 631 thereof in an upright position, so as to further overcome the problem that the liquid-conducting element 63 and the free end 631 thereof tilt or deform and loosen, and to prevent liquid from seeping into the interior of the atomizer to contaminate the atomizer.

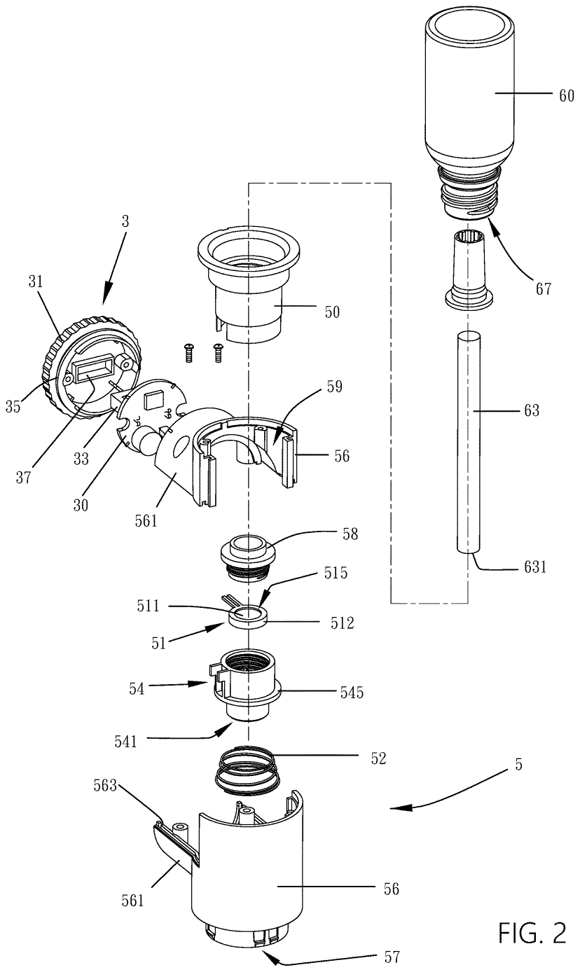

Please refer to FIGS. 2 to 4, In some embodiments of the ultrasonic atomizer using a liquid bottle of the present invention, the atomizing module 5 further comprises an elastic member 52, the elastic member 52 being compressed and coupled between in the upper side of the hollow housing 56 and the upper side of the mist-guiding tube 54 while storing tension. By virtue of one side of the mist-guiding tube 54 at the mist outlet being movably coupled in the first opening 57 in the hollow housing 56, the upper side of the connecting tube 58 being coupled in the lower side of the mist-guiding tube 54, and the lower side of the connecting tube 58 being movably connected to the upper side of the assembling seat 50, the tension of the elastic member 52 is used to movably drive the mist-guiding tube 54 and the connecting tube 58 downward, while the free end of the inwardly protruding tube 543 pushes downward against the piezoelectric vibration element 511 of the piezoelectric vibration module 51 so as to further move downward and more tightly abut against the free end 631 of the liquid-conducting element 63 protruding from the bottle opening of the liquid bottle 60, so that the liquid (such as essential oil or aromatic liquid) at the free end 631 of the liquid-conducting element 63 can be more tightly and effectively transmitted to the piezoelectric vibration element 511 for atomization.

In some embodiments, a gap 501 is predetermined between the lower side of the connecting tube 58 and the upper side of the assembling seat 50. The tension of the elastic member 52 drives the mist-guiding tube 54 and the connecting tube 58 downward, while the free end of the inwardly protruding tube 543 pushes downward against the piezoelectric vibration element 511 of the piezoelectric vibration module 51 so that the piezoelectric vibration element 511 moves downward within the distance of the gap 501, so as to movably adjust the downward abutting contact of the piezoelectric vibration element 511 against the free end 631 of the liquid-conducting element 63. By means of the above embodiment of the ultrasonic atomizer using a liquid bottle of the present invention, the piezoelectric vibration element 511 of the piezoelectric vibration module 51 is allowed to continuously and effectively contact, in a downward manner, the essential oil or liquid at the free end 631 of the liquid-conducting element 63, so as to maintain a proper liquid film thickness at the atomization interface between the free end of the liquid-conducting element 63 and the piezoelectric vibration element 511 and effectively atomize the liquid.

Please refer to FIGS. 2 to 4, In some embodiments of the ultrasonic atomizer using a liquid bottle of the present invention, the piezoelectric vibration module 51 comprises a fixing sleeve 512, the fixing sleeve being provided with an opening 515. The fixing sleeve 512 may be made of silicone, plastic, rubber, or any of various waterproof or insulating materials. In the illustrated embodiment, the piezoelectric vibration element 511 is, for example, a truncated-cone-shaped vibration sheet made of nickel alloy or iron alloy, and the plurality of micro-holes 513 are formed at a center of the truncated-cone-shaped vibration sheet. An annular piezoelectric ceramic plate may be coupled to the truncated-cone-shaped vibration sheet, and an opening of the annular piezoelectric ceramic plate passes around the outer periphery of the plurality of micro-holes 513, so that the plurality of micro-holes 513 are located within the opening of the annular piezoelectric ceramic plate and are assembled as the piezoelectric vibration element 511. The piezoelectric vibration element 511 is disposed in the fixing sleeve 512, the plurality of micro-holes 513 of the piezoelectric vibration element 511 correspond to the opening 515 of the fixing sleeve 512, the upper side of the opening 515 is connected to the free end of the inwardly protruding tube 543, and the lower side of the opening 515 is connected to the upper side of the connecting tube 58.

Please refer to FIGS. 2 to 4, In some embodiments of the ultrasonic atomizer using a liquid bottle of the present invention, the inwardly protruding tube 543 is formed as a tapered tube structure that gradually narrows downward from the inner side of the mist outlet 541, and a diameter of a free end of the tapered tube structure of the inwardly protruding tube 543 is the same as, or smaller than, the diameter of the opening 515 of the fixing sleeve 512, such that the inwardly protruding tube 543 can be accurately connected to the opening 515, so as to deliver the atomized droplets generated by oscillation of the piezoelectric vibration element 511 to the mist outlet 541 without leakage to the outside. the inner surface of the tapered tube structure of the inwardly protruding tube 543 is formed with a gradually tapered slope 545 to the free end of the inwardly protruding tube 543, whereby atomized droplets in the mist outlet 541, a small portion of droplets may do not completely exit from the mist outlet 541 and flow back downward, to be collected by the slope 545 to the piezoelectric vibration element 511 for oscillation and atomization again, so as to effectively use the liquid (such as essential oil or aromatic liquid).

In some embodiments, the diameter of the free end 631 of the liquid-conducting element 63 is smaller than the diameter of the opening 515 of the fixing sleeve 512, whereby the free end 631 of the liquid-conducting element 63 can be coupled in the opening 515 and contact the piezoelectric vibration element 511 and the plurality of micro-holes 513 thereof in a circumferentially confined manner, so as to more effectively deliver liquid to the piezoelectric vibration element 511.

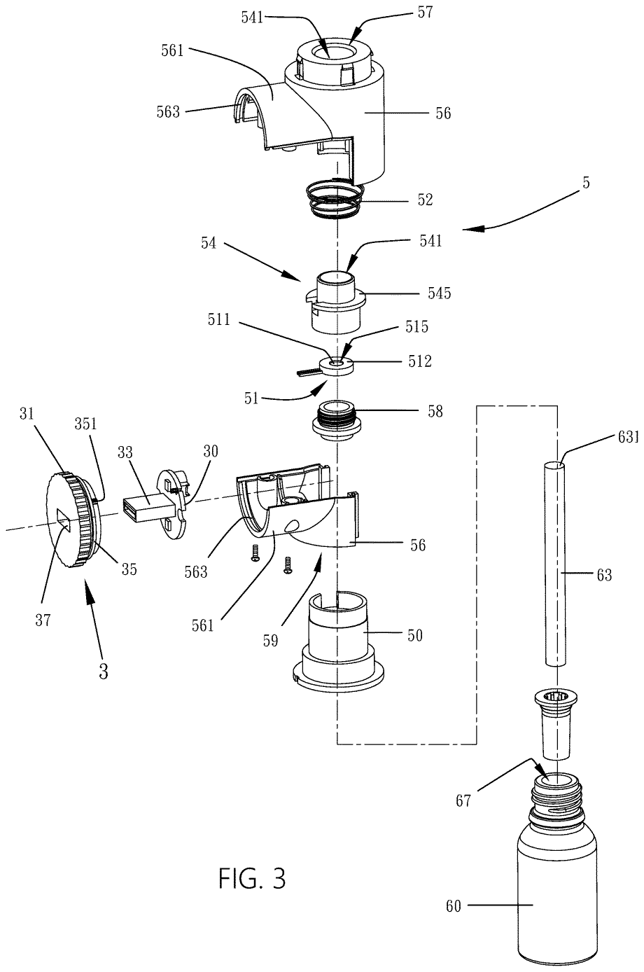

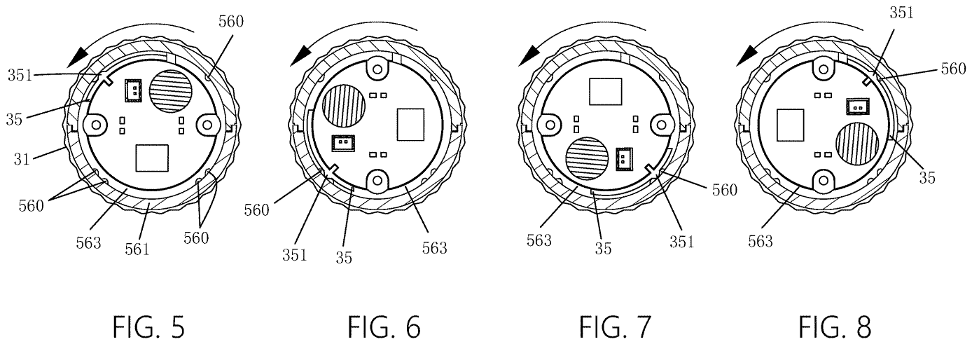

Please refer to FIGS. 3 to 5, In some embodiments of the ultrasonic atomizer using a liquid bottle of the present invention, the power plug 3 having a rotatable angle-changing structure, and is configured to plug the power plug 3 into an electrical socket in various different orientations. In the embodiments, the power plug 3 is rotatably assembled to the atomizing module 5 so that its angle can be changed. The power plug 3 is configured to be detachably coupled to a USB electrical socket (not show in the drawings) in different orientations, such that the ultrasonic atomizer using a liquid bottle of the present invention can rotate by an angle relative to the USB electrical socket, for example, 1 degree to 360 degrees, whereby the atomizer and the liquid bottle 60 can be maintained upward during use for atomization. The power plug 3 comprises a rotating portion 31 and a USB plug 33. One side of the rotating portion 31 is provided with a protruding rail 35 extending in parallel, and the rotating portion 31 is formed with a plug hole 37. The USB plug 33 may be, for example, a Type-A, Type-C, or Micro-USB plug. The USB plug 33 is coupled in the plug hole 37 to form the rotatable power plug 3. One side of the hollow housing 56 extends with a connecting pipe 561, an inner side of the connecting pipe 561 is formed with a concave rail 563, and the protruding rail 35 of the rotating portion 31 is rotatably coupled in the concave rail 563 on the inner side of the connecting pipe 561. Thereby, when the ultrasonic atomizer using a liquid bottle of the present invention uses the USB plug 33 to be electrically connected in a USB electrical socket (not show in the drawings) of an unfavorable orientation, the ultrasonic atomizer using a liquid bottle can rotate by an angle through the rotating portion 31 and the USB plug 33 relative to the USB electrical socket, so that the atomizer and the liquid bottle 60 can be maintained at an upward angle during use, thereby overcoming the problem that an USB plug of the atomizer is electrically connected for use with USB electrical sockets arranged in different orientations.

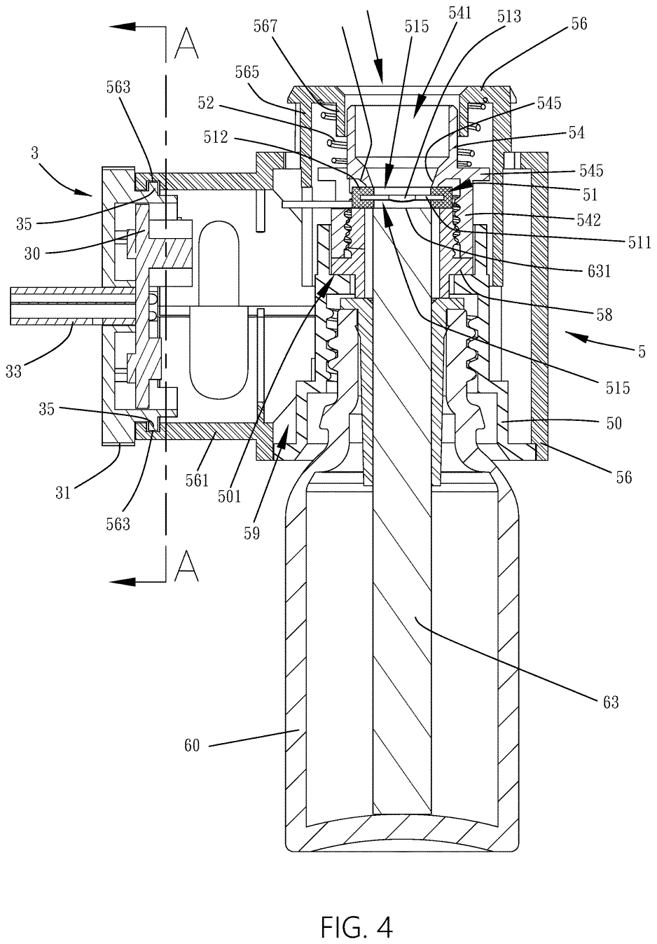

Please refer to FIGS. 4 to 8, In some embodiments of the ultrasonic atomizer using a liquid bottle of the present invention, the protruding rail 35 of the rotating portion 31 may be configured to be tightly fitted in the concave rail 563 of the connecting pipe 561, wherein a damping or frictional effect generated when the protruding rail 35 and the concave rail 563 relatively rotate is used to movably fix the connection angle between the power plug 3 and the hollow housing 56, for example including but not limited to 90 degrees or 180 degrees or 270 degrees.

In some embodiments, a protrusion 351 is formed on the surface of the protruding rail 35, and correspondingly, stopper 560 is provided at a 90 degrees or 180 degrees or 270 degrees position on either side of the concave rail 563, whereby when the rotating portion 31 drives the protruding rail 35 and the protrusion 351 thereof to rotate to one side in a 90 degrees or 180 degrees or 270 degrees direction, the protrusion 351 is movably engaged with the stopper 560, please refer to FIG. 6 is a schematic view showing that, when the power plug 3 is rotated by 90 degrees, the protrusion 351 of the protruding rail 35 is movably engaged with the stoppers 560 of concave rail 563, FIG. 7 is a schematic view showing that, when the power plug 3 is rotated by 180 degrees, the protrusion 351 of the protruding rail 35 is movably engaged with the stoppers 560 of the concave rail 563, and FIG. 8 is a schematic view showing that, when the power plug 3 is rotated by 270 degrees, the protrusion 351 of the protruding rail 35 is movably engaged with the stoppers 560 of concave rail 563.

The rotatable-angle power plug 3 can rotate in 90 degrees or 180 degrees or 270 degrees direction relative to the mist outlet 541 and the liquid bottle 60 coupled with the hollow housing 56, whereby the power plug 3 after being rotated by a degree can be plugged into an electrical socket of an unfavorable orientation (not shown in drawings), thereby completing the function that the ultrasonic atomizer using a liquid bottle of the present invention can rotate by a degrees relative to the electrical socket.

Please refer to FIGS. 1 to 4, In some embodiments of the ultrasonic atomizer using a liquid bottle of the present invention, the upper side of the hollow housing 56 has an outer wall 565 and an inner wall 567, the inner wall 567 being formed in parallel on the inner side of the outer wall 565, the inner space of the inner wall 567 defining the first opening 57. A protruding rib 545 is formed on the outer side of the mist-guiding tube 54. The elastic member 52 comprises any one of a spring, an elastic block, or an elastic plate, and in the embodiments the elastic member 52 is a spring. One side of the spring storing tension is sleeved around the outer periphery of the inner wall 567, and another side of the spring is sleeved on the upper side of the mist-guiding tube 54 and abuts against the protruding rib 545, whereby the spring tension can drive the mist-guiding tube 54 and the connecting tube 58 downward, and simultaneously drive the inwardly protruding tube 543 to push the piezoelectric vibration element 511 of the piezoelectric vibration module 51 downward to tightly abut against the free end 631 of the liquid-conducting element 63.

What the invention claimed is:

1. An ultrasonic atomizer using a liquid bottle, comprising: a power plug, a control circuit, and an atomizing module, wherein the atomizing module comprises a piezoelectric vibration module, a mist-guiding tube, a hollow housing, a connecting tube, and an assembling seat;
wherein the mist-guiding tube comprises an mist outlet on the upper side thereof and an inwardly protruding tube formed by downward protrusion from the inner side of the mist outlet, and the hollow housing is formed with a first opening at the upper side thereof and a second opening at the lower side thereof;
wherein one side of the mist-guiding tube at the mist outlet is movably coupled in the first opening in the hollow housing;
wherein the piezoelectric vibration module comprises a piezoelectric vibration element having plurality of micro-holes formed thereon, the piezoelectric vibration module is coupled in the mist-guiding tube, the upper side of the piezoelectric vibration module is connected to the free end of the inwardly protruding tube, the upper side of the connecting tube is coupled in the lower side of the mist-guiding tube, and the upper free end of the connecting tube is connected to the lower side of the piezoelectric vibration module, whereby the plurality of micro-holes of the piezoelectric vibration element, the inwardly protruding tube, the mist outlet, and the connecting tube form a communicating passage;
wherein the assembling seat is coupled in the second opening, and the lower side of the connecting tube is movably connected to the upper side of the assembling seat;
wherein the power plug is coupled to one side of the hollow housing, the power plug is electrically connected to the control circuit, the control circuit is electrically connected to the piezoelectric vibration element of the piezoelectric vibration module.

2. The ultrasonic atomizer using a liquid bottle as claimed in claim 1, further comprising a liquid bottle, wherein the liquid bottle is detachably coupled to the assembling seat, the liquid bottle having a bottle opening and a liquid-conducting element, one side of the liquid-conducting element is disposed in the liquid within the liquid bottle, and another side thereof extends outward from the bottle opening and passes through the connecting tube, and the free end of the liquid-conducting element protrudes from the upper side of the connecting tube so as to be connected the lower side of the piezoelectric vibration element.

3. The ultrasonic atomizer using a liquid bottle as claimed in claim 2, wherein the liquid-conducting element comprises any one of a cotton wick, a PET fiber rod, a paper rod, a foam rod, or a porous liquid-conducting element made of a material having a porous structure or capillary action.

4. The ultrasonic atomizer using a liquid bottle as claimed in claim 2, wherein the atomizing module further comprises an elastic member, the elastic member being compressed and coupled between in the upper side of the hollow housing and the upper side of the mist-guiding tube while storing tension, whereby, the tension of the elastic member is used to movably drive the mist-guiding tube and the connecting tube downward, while the free end of the inwardly protruding tube pushes downward against the piezoelectric vibration element of the piezoelectric vibration module so as to further move downward and more tightly abut against the free end of the liquid-conducting element protruding from the bottle opening of the liquid bottle.

5. The ultrasonic atomizer using a liquid bottle as claimed in claim 4, wherein a gap is predetermined between the lower side of the connecting tube and the upper side of the assembling seat, and the tension of the elastic member drives the mist-guiding tube and the connecting tube downward, while the free end of the inwardly protruding tube pushes downward against the piezoelectric vibration element of the piezoelectric vibration module so that the piezoelectric vibration element moves downward within a distance of the gap, so as to movably adjust the downward abutting contact of the piezoelectric vibration element against the free end of the liquid-conducting element.

6. The ultrasonic atomizer using a liquid bottle as claimed in claim 1, wherein the piezoelectric vibration module comprises a fixing sleeve, the fixing sleeve is provided with an opening, the piezoelectric vibration element is disposed in the fixing sleeve, the plurality of micro-holes of the piezoelectric vibration element correspond to the opening of the fixing sleeve, the upper side of the opening is connected to the free end of the inwardly protruding tube, and the lower side of the opening is connected to the upper side of the connecting tube.

7. The ultrasonic atomizer using a liquid bottle as claimed in claim 6, wherein the piezoelectric vibration element comprises a truncated-cone-shaped vibration sheet made of nickel alloy or iron alloy, the plurality of micro-holes are formed at the center of the truncated-cone-shaped vibration sheet, and an annular piezoelectric ceramic plate is coupled to the truncated-cone-shaped vibration sheet, an opening of the annular piezoelectric ceramic plate passing around the outer periphery of the plurality of micro-holes, so that the plurality of micro-holes are located within the opening of the annular piezoelectric ceramic plate and are assembled as the piezoelectric vibration element.

8. The ultrasonic atomizer using a liquid bottle as claimed in claim 6, wherein the inwardly protruding tube is formed as a tapered tube structure that gradually narrows downward from the inner side of the mist outlet, and the diameter of a free end of the tapered tube structure of the inwardly protruding tube is the same as, or smaller than, the diameter of the opening of the fixing sleeve, such that the inwardly protruding tube can be accurately connected to the opening.

9. The ultrasonic atomizer using a liquid bottle as claimed in claim 8, wherein the inner surface of the tapered tube structure of the inwardly protruding tube is formed with a gradually tapered slope to the free end of the inwardly protruding tube.

10. The ultrasonic atomizer using a liquid bottle as claimed in claim 6, wherein the diameter of the free end of the liquid-conducting element is smaller than the diameter of the opening of the fixing sleeve, whereby the free end of the liquid-conducting element can be coupled in the opening and contact the piezoelectric vibration element and the plurality of micro-holes thereof in a circumferentially confined manner.

11. The ultrasonic atomizer using a liquid bottle as claimed in claim 1, wherein the power plug is rotatably assembled to the atomizing module so that an angle thereof can be changed, the power plug comprises a rotating portion and a USB plug, one side of the rotating portion is provided with a protruding rail extending in parallel, and the rotating portion is formed with a plug hole, the USB plug being coupled in the plug hole; one side of the hollow housing extends with a connecting pipe, the inner side of the connecting pipe is formed with a concave rail, and the protruding rail of the rotating portion is rotatably coupled in the concave rail on the inner side of the connecting pipe.

12. The ultrasonic atomizer using a liquid bottle as claimed in claim 11, wherein the power plug is configured to be detachably coupled to a USB electrical socket in different orientations, and the ultrasonic atomizer using a liquid bottle is rotatable by an angle relative to the USB electrical socket, whereby the atomizer and the liquid bottle can be maintained vertically upward for atomization during use.

13. The ultrasonic atomizer using a liquid bottle as claimed in claim 11, wherein a damping or frictional effect generated when the protruding rail and the concave rail relatively rotate is used to movably fix a connection angle between the power plug and the hollow housing.

14. The ultrasonic atomizer using a liquid bottle as claimed in claim 11, wherein a protrusion is formed on a surface of the protruding rail, and correspondingly, stopper is formed at a 90 degrees or 180 degrees or 270 degrees position on either side of the concave rail, whereby when the rotating portion drives the protruding rail and the protrusion thereof to rotate to one side in a 90 degrees or 180 degrees or 270 degrees direction, the protrusion is movably engaged with the stopper.

15. The ultrasonic atomizer using a liquid bottle as claimed in claim 11, wherein the upper side of the hollow housing has an outer wall and an inner wall, the inner wall being formed in parallel on the inner side of the outer wall, the inner space of the inner wall defining the first opening; wherein a protruding rib is formed on an outer side of the mist-guiding tube, the elastic member storing tension and one side of the elastic member being sleeved around the outer periphery of the inner wall, and another side of the elastic member being sleeved on the upper side of the mist-guiding tube and abutting against the protruding rib.

16. The ultrasonic atomizer using a liquid bottle as claimed in claim 1, wherein the control circuit comprises an oscillator circuit and a driver circuit.
